(19) United States
(12) Patent Application Publication
(10) Pub. No.: US 2013/0158619 A1
(43) Pub. Date: Jun. 20, 2013

(54) MULTI-VENTRICULAR SITE TIMING OPTIMIZATION USING CARDIOGENIC IMPEDANCE

(71) Applicants: Kwangdeok Lee, Valencia, CA (US); Jong Gill, Valencia, CA (US); Kyungmoo Ryu, Palmdale, CA (US)

(72) Inventors: Kwangdeok Lee, Valencia, CA (US); Jong Gill, Valencia, CA (US); Kyungmoo Ryu, Palmdale, CA (US)

(73) Assignee: PACESETTER, INC., Sylmar, CA (US)

(21) Appl. No.: 13/329,091

(22) Filed: Dec. 16, 2011

(51) Int. Cl. A61N 1/365

(52) U.S. Cl. 607/17

(57) ABSTRACT

A method of calculating a timing delay for an implantable medical device based on cardiogenic impedance estimates cardiogenic impedance from a signal between a first electrode and a second electrode positioned in at least one chamber of a heart. The method also determines the timing delay based on the estimated cardiogenic impedance.

### FIELD

The present disclosure is related, generally, to cardiac resynchronization therapy and, more specifically to multi-ventricular site timing optimization or improvement using cardiogenic impedance.

### BACKGROUND

A current solution for cardiac resynchronization therapy (CRT) timing optimization is based on echocardiography (echo) techniques, e.g., Doppler echo techniques. However, timing optimization using Doppler echo techniques is not commonly performed in routine clinical practices due to time and cost of these procedures. Moreover, not every center is equipped to efficiently perform complex echo measures such as tissue Doppler imaging (TDI). Because the timing delays change over time, frequent re-optimization of these delays would be beneficial.

Another technique involves an intracardiac electrogram (IEGM) optimization method, which can estimate the optimal paced/sensed atrio-ventricular (AV) and interventricular (V-V) delays. This IEGM optimization method correlates well with echo-based optimization. The IEGM optimization method may be based on programmed inputs implemented within a short time period.

The IEGM optimization method, however, is not available in patients lacking intrinsic conduction. A significant number of heart failure patients do not have intrinsic conduction, including patients with sinus node dysfunction, atrioventricular (AV) block and/or atrial fibrillation. Moreover, the existing IEGM solutions do not address left ventricular to left ventricular (LV-LV) (i.e., intra-ventricular) delay associated with multisite LV leads.

Furthermore, mechanical and electrical dyssynchrony are not well correlated in heart failure patients. Therefore, it would be desirable to have a surrogate for mechanical dyssynchrony to improve paced/sensed AV, V-V, and LV-LV delay optimization.

### SUMMARY

According to an aspect of the present disclosure, a method calculates a timing delay for an implantable medical device based on cardiogenic impedance. The method estimates cardiogenic impedance from a signal between a first electrode and a second electrode positioned in at least one chamber of a heart. The method also determines the timing delay based on the estimated cardiogenic impedance.

According to another aspect, an apparatus calculates a timing delay for an implantable medical device based on cardiogenic impedance. The apparatus includes a memory and at least one processor coupled to the memory and configured to estimate cardiogenic impedance from a signal between a first electrode and a second electrode positioned in at least one chamber of a hear. The processor(s) is also configured to determine the timing delay based on the estimated cardiogenic impedance.

In yet another aspect, an apparatus calculates a timing delay for an implantable medical device based on cardiogenic impedance. The apparatus has means for estimating cardiogenic impedance from a signal between a first electrode and a second electrode positioned in at least one chamber of a heart. The apparatus also has means for determining the timing delay based on the estimated cardiogenic impedance.

In still another aspect, a computer program product for calculating a timing delay for an implantable medical device based on cardiogenic impedance includes a computer-readable medium having non-transitory program code recorded thereon. The program code includes program code to estimate cardiogenic impedance from a signal between a first electrode and a second electrode positioned in at least one chamber of a heart. The program code also includes program code to determine the timing delay based on the estimated cardiogenic impedance.

This has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF FIGURES

The features, nature, and advantages of the present disclosure will become more apparent from the detailed description set forth below when taken in conjunction with the drawings in which like reference characters identify correspondingly throughout.

FIG. 1 schematically illustrates an exemplary implantable medical device (IMD) in electrical communication with the heart of a patient.

FIG. 2 schematically illustrates an exemplary implantable stimulation device configured as a system according to some aspects of the disclosure.

FIG. 3 illustrates a correlation of a cardiogenic impedance feature with left atrial pressure.

FIG. 4 is an exemplary schematic diagram illustrating the effect of atrioventricular delay duration on Doppler echocardiographic recordings of transmitral flow.

FIG. 5 illustrates sample cardiogenic impedance signal with respect to time delay according to some aspects of the disclosure.

FIG. 6 illustrates a comparison between stroke volume and right ventricular peak-to-peak resistance according to some aspects of the disclosure

FIG. 7 illustrates an exemplary flowchart of a method for multi-ventricular site timing optimization based on cardiogenic impedance, according to one aspect of the present disclosure.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion. The following description includes the best mode presently contemplated for practicing the present teachings. The description is not to be taken in a limiting sense but is merely for the purpose of describing the general principles of the illustrative embodiments. The scope of the present teachings should be ascertained with reference to the claims. In the description that follows, like numerals or reference designators will refer to like parts or elements throughout.

Some portions of the following detailed description are presented in terms of algorithms and symbolic representations of operations on data bits or binary digital signals within a computer memory. These algorithmic descriptions and representations may be the techniques used by those skilled in the data processing arts to convey the substance of their work to others skilled in the art.

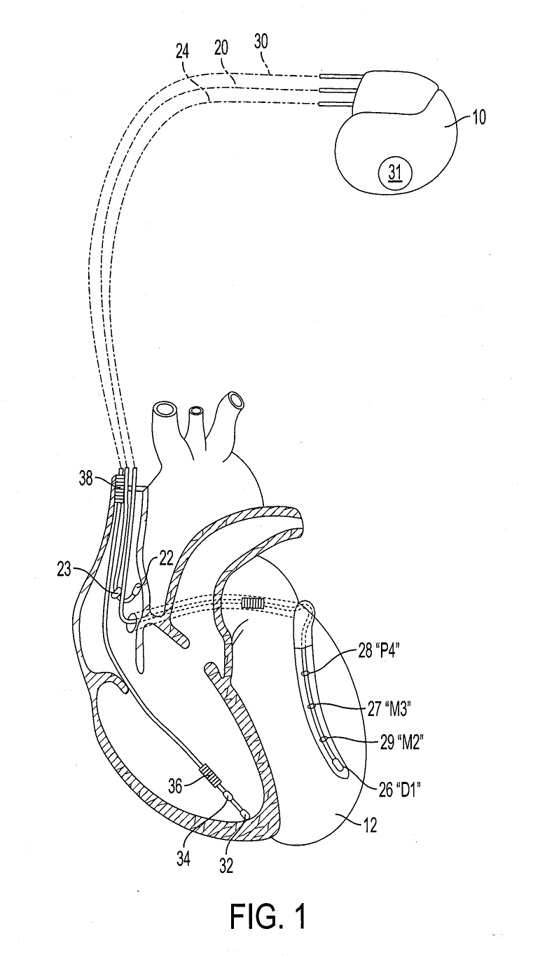

With reference to FIG. 1, there is a stimulation device or IMD 10 in electrical communication with the heart 12 of a patient by way of three leads, 20, 24 and 30, suitable for delivering multi-chamber stimulation and shock therapy. To sense atrial cardiac signals and to provide right atrial chamber stimulation therapy, the stimulation device 10 is coupled to an implantable right atrial lead 20 having at least an atrial tip electrode 22, which typically is implanted in the right atrial appendage, and an atrial ring electrode 23.

To sense left atrial and ventricular cardiac signals and to provide left chamber pacing therapy, the stimulation device 10 is coupled to a quad pole lead 24 designed for placement in the latero or postero-lateral branch of the left ventricle via the coronary sinus. Accordingly, an exemplary quad pole lead 24 is designed to receive atrial and ventricular cardiac signals and to deliver left heart pacing therapy using at least a left ventricular distal electrode (D1) 26, mid first ring (M2) 29, mid second ring (M3) 27 and proximal ring (P4) 28. The inter-electrode spacing, in one embodiment, is 20 mm (D1-M2), 10 mm (M2-M3), and 17 mm (M3-P4). Thus, from tip to proximal the lead spans 47 mm. When the tip is pushed as far as anatomically possible in a coronary sinus branch, the proximal ring is often near the atrial-ventricular (AV) groove and sometimes even in the main coronary sinus or Great Cardiac Vein instead of the branch. The unipolar P4-RV coil sense vector, the bipolar M3-P4 sense vector, and sometimes additional unipolar and bipolar vectors, display both atrial and ventricular potentials on the electrogram. In one embodiment, the mid second ring (M3) 27 and the proximal ring (P4) 28 represent electrical signals of the left atrium.

As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and shocking therapy using at least a left atrial coil electrode 28.

The stimulation device 10 is also shown in electrical communication with the heart by way of an implantable right ventricular lead 30 having, in this embodiment, a right ventricular tip electrode 32, a right ventricular ring electrode 34, a right ventricular (RV) coil electrode 36, and a superior vena cava (SVC) coil electrode 38. Typically, the right ventricular lead 30 is transvenously inserted into the heart to place the right ventricular tip electrode 32 in the right ventricular apex so the RV coil electrode 36 is positioned in the right ventricle and the SVC coil electrode 38 is positioned in the superior vena cava. Accordingly, the right ventricular lead 30 is capable of receiving cardiac signals, and delivering stimulation in the form of pacing and shock therapy to the right ventricle. To provide a “vibratory alert” signal (from a motor with an offset mass that can be provided in the device can), an additional electrode 31 can be provided in proximity to the device.

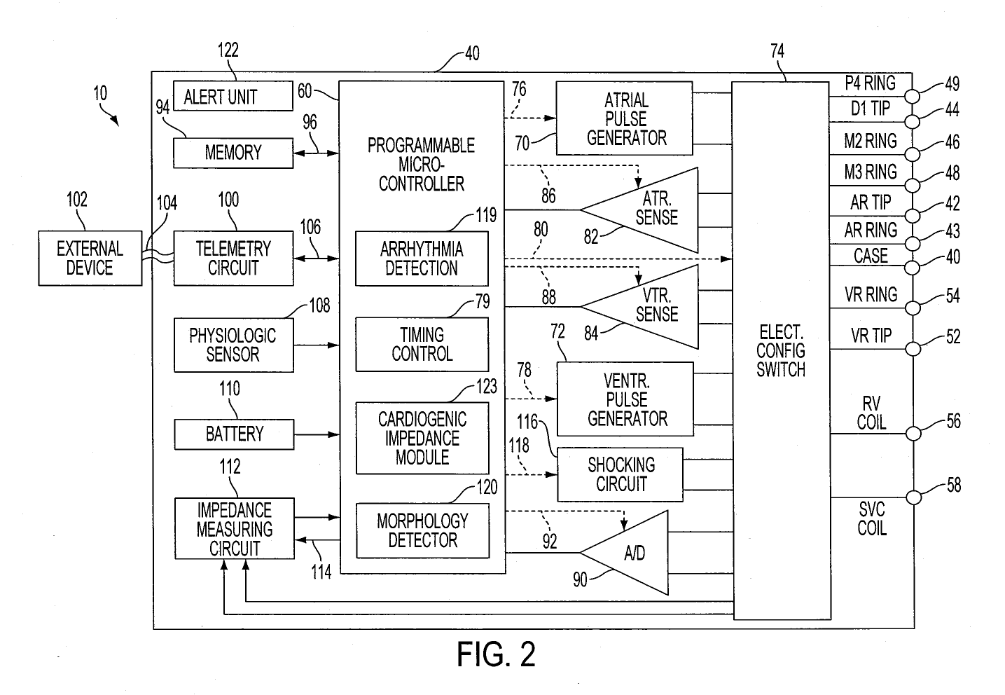

As illustrated in FIG. 2, a simplified block diagram is shown of the multi-chamber implantable stimulation device 10, which is capable of treating both fast and slow arrhythmias with stimulation therapy, including cardioversion, defibrillation, and pacing stimulation. The stimulation device 10 is configured as a system in which the various embodiments of the present teachings may operate. While a particular multi-chamber device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation.

The housing 40 for the stimulation device 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the coil electrodes 36 and 38, (FIG. 1) for shocking purposes. The housing 40 further includes a connector (not shown) having terminals, 42, 43, 44, 46, 48, 49, 52, 54, 56 and 58 (shown schematically and, for convenience, the names of the electrodes to which they are connected are shown next to the terminals).

As such, to achieve right atrial sensing and pacing, the connector includes at least a right atrial tip terminal (AR TIP) 42 adapted for connection to the atrial tip electrode 22(FIG. 1) and a right atrial ring (AR RING) electrode 43 adapted for connection to the right atrial ring electrode 23 (FIG. 1). To achieve left chamber sensing and pacing, the connector includes at least a left ventricular tip terminal (D1 TIP) 44, a left ventricular ring terminal (M2 RING) 46, a left heart ring terminal (M3 RING) 48, and a left heart proximal terminal (P4 RING) 49, which are adapted for connection to the left ventricular distal electrode (D1) 26 (FIG. 1), the mid first ring (M2) 29 (FIG. 1), the mid second ring (M3) 27 (FIG. 1) and the proximal ring (P4) 28 (FIG. 1), respectively. As noted previously, the mid second ring (M3) 27 and the proximal ring (P4) 28 are possibly located in the left ventricle or the left atrium.

To support right chamber sensing, pacing and shocking, the connector further includes a right ventricular tip terminal (VR TIP) 52, a right ventricular ring terminal (VR RING) 54, a right ventricular shocking terminal (RV COIL) 56, and an SVC shocking terminal (SVC COIL) 58, which are adapted for connection to the right ventricular tip electrode 32 (FIG. 1), right ventricular ring electrode 34 (FIG. 1), the RV coil electrode 36 (FIG. 1), and the SVC coil electrode 38 (FIG. 1), respectively. To provide the “vibratory alert” signal, a vibratory alert unit 122 generates a signal for an additional terminal (not shown) for connection to the vibratory alert electrode 31 (FIG. 1). In one embodiment, the vibratory alert will alert the patient, and then a home monitor can be used to transfer the information associated with the alert from the device 10 to an attending medical professional, who can take the appropriate clinical action.

At the core of the stimulation device 10 is a programmable microcontroller 60, which controls the various modes of stimulation therapy. As is well known in the art, the microcontroller 60 (also referred to as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. The microcontroller 60 includes the ability to process or monitor input signals (data) as controlled by program code stored in a designated block of the memory. The details of the design and operation of the microcontroller 60 are not critical to the present teachings. Rather, any suitable microcontroller 60 may be used that carries out the functions described. The use of microprocessor-based control circuits for performing timing and data analysis functions are well known in the art.

As shown in FIG. 2, an atrial pulse generator 70 and a ventricular pulse generator 72 generate pacing stimulation pulses for delivery by the right atrial lead 20 (FIG. 1), the right ventricular lead 30 (FIG. 1), and/or the quad pole lead 24 (FIG. 1) via an electrode configuration switch 74. It is understood that in order to provide stimulation therapy in each of the four chambers of the heart, the atrial and ventricular pulse generators, 70 and 72, may include dedicated, independent pulse generators, multiplexed pulse generators or shared pulse generators. The pulse generators, 70 and 72, are controlled by the microcontroller 60 via appropriate control signals, 76 and 78, respectively, to trigger or inhibit the stimulation pulses.

The microcontroller 60 further includes timing control circuitry 79 that controls the timing of such stimulation pulses (e.g., pacing rate, atrioventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., as is well known in the art. The switch 74 includes multiple switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, combipolar, etc.) by selectively closing the appropriate combination of switches (not shown) as is known in the art.

Atrial sensing circuits 82 and ventricular sensing circuits 84 may also be selectively coupled to the right atrial lead 20 (FIG. 1), the quad pole lead 24 (FIG. 1), and the right ventricular lead 30 (FIG. 1), through the switch 74 for detecting the presence of cardiac activity in each of the four chambers of the heart. Accordingly, the atrial (ATR. SENSE) and ventricular (VTR. SENSE) sensing circuits, 82 and 84, may include dedicated sense amplifiers, multiplexed amplifiers or shared amplifiers and may receive control signals 86, 88 from the controller 60. The switch 74 determines the “sensing polarity” of the cardiac signal by selectively closing the appropriate switches, as is also known in the art. In this way, the clinician may program the sensing polarity independent of the stimulation polarity. Each sensing circuit, 82 and 84, preferably employs one or more low power, precision amplifiers with programmable gain and/or automatic gain control, band pass filtering, and a threshold detection circuit, as known in the art, to selectively sense the cardiac signal of interest. The automatic gain control enables the device 10 to effectively address the difficult problem of sensing the low amplitude signal characteristics of atrial or ventricular fibrillation. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart.

For arrhythmia detection, the device 10 utilizes the atrial and ventricular sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks).

Cardiac signals are also applied to the inputs of an analog-to-digital (A/D) data acquisition system 90. The data acquisition system 90 is configured to acquire intra-cardiac electrogram (IEGM) signals, convert the raw analog data into a digital signal, and store the digital signals for later processing and/or telemetric transmission to an external device 102. The data acquisition system 90 is coupled to the right atrial lead 20 (FIG. 1), the quad pole lead 24 (FIG. 1), and the right ventricular lead 30 (FIG. 1) through the switch 74 to sample cardiac signals across any pair of desired electrodes. The controller 60 controls the data acquisition system via control signals 92.

The microcontroller 60 is further coupled to a memory 94 by a suitable data/address bus 96. The programmable operating parameters used by the microcontroller 60 are stored and modified, as required, in order to customize the operation of the IMD deice 10 to suit the needs of a particular patient. The memory 94 includes software modules, such as a cardiogenic impedance module 123, which, when executed or used by the microcontroller 60, provides the operational functions of the implantable medical device 10. Additional operating parameters and code stored on the memory 94 define, for example, pacing pulse amplitude or magnitude, pulse duration, electrode polarity, rate, sensitivity, automatic features, arrhythmia detection criteria, and the amplitude, wave shape and vector of each shocking pulse to be delivered to the patient's heart within each respective tier of therapy. Other pacing parameters include base rate, rest rate and circadian base rate.

Advantageously, the operating parameters of the implantable device 10 may be non-invasively programmed into the memory 94 through a telemetry circuit 100 in telemetric communication with the external device 102, such as a programmer, trans-telephonic transceiver, a diagnostic system analyzer, or even a cellular telephone. The telemetry circuit 100 is activated by the microcontroller by a control signal 106. The telemetry circuit 100 advantageously allows intra-cardiac electrograms and status information relating to the operation of the device 10 (as contained in the microcontroller 60 or memory 94) to be sent to the external device 102 through an established communication link 104. In one embodiment, the stimulation device 10 further includes a physiologic sensor 108, commonly referred to as a “rate-responsive” sensor because it adjusts pacing stimulation rate according to the exercise state of the patient. However, the physiological sensor 108 may further be used to detect changes in cardiac output, changes in the physiological condition of the heart, or diurnal changes in activity (e.g., detecting sleep and wake states). Accordingly, the microcontroller 60 responds by adjusting the various pacing parameters (such as rate, AV Delay, V-V Delay, etc.) at which the atrial and ventricular pulse generators, 70 and 72, generate stimulation pulses. While shown as being included within the stimulation device 10, it is to be understood that the physiologic sensor 108 may also be external to the stimulation device 10, yet still be implanted within or carried by the patient.

The stimulation device additionally includes a battery 110, which provides operating power to all of the circuits shown in FIG. 2. For the stimulation device 10, which employs shocking therapy, the battery 110 is capable of operating at low current drains for long periods of time, and is capable of providing high-current pulses (for capacitor charging) when the patient requires a shock pulse. The battery 110 also has a predictable discharge characteristic so that elective replacement time can be detected. In one embodiment, the device 10 employs lithium/silver vanadium oxide batteries. As further shown in FIG. 2, the device 10 has an impedance measuring circuit 112 enabled by the microcontroller 60 via a control signal 114.

When the stimulation device 10 is intended to operate as an IMD, it detects the occurrence of an arrhythmia and automatically applies an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 joules), as controlled by the microcontroller 60. Such shocking pulses are applied to the heart 12 through at least two shocking electrodes, and as shown in this embodiment, selected from the RV coil electrode 36 (FIG. 1), the SVC coil electrode 38 (FIG. 1) and the case 10. Cardioversion shocks are generally considered to be of low to moderate energy level (to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (because R-waves may be too disorganized), and pertaining exclusively to the treatment of fibrillation. Accordingly, the microcontroller 60 is capable of controlling the synchronous or asynchronous delivery of the shocking pulses.

The microcontroller 60 includes a morphology detector 120 for tracking various morphological features within electrical cardiac signals, including intervals between polarization events, elevations between polarization events, durations of polarization events and amplitudes of polarization events. The microcontroller 60 also includes an arrhythmia detection control 119 that analyzes the sensed electrical signals to determine whether arrhythmia is being experienced. A cardiogenic impedance module 123, in cooperation with the memory 94, assists in monitoring cardiogenic impedance.

The remaining figures, flow charts, graphs and other diagrams illustrate the operation and novel features of the stimulation device 10 as configured in accordance with exemplary embodiments of the present teachings. In the flow chart, the various process steps are summarized in individual “blocks.” Such blocks describe specific actions or decisions made or carried out as the process proceeds. Where a microcontroller (or equivalent) is employed, the functional block diagrams provide the basis for timing optimization using cardiogenic impedance that may be used by such a microcontroller (or equivalent). Those skilled in the art may readily write such a program based on the functional block diagrams and other descriptions presented herein.

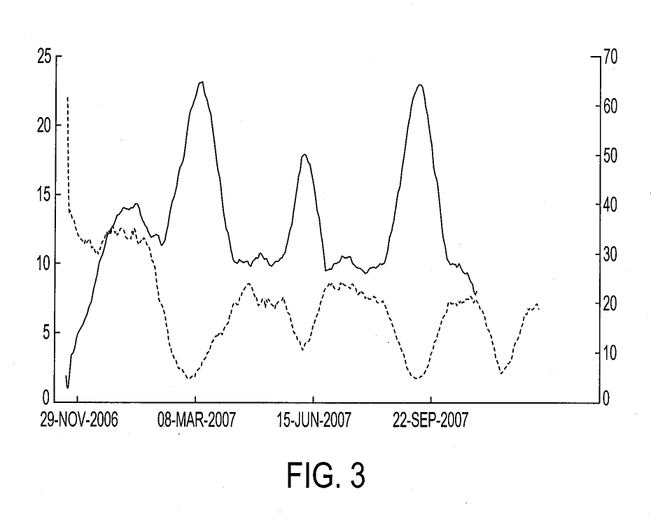

Cardiogenic impedance (CI) features have been shown to correlate well with hemodynamic parameters such as cardiac contractility. It has also been shown that a cardiogenic impedance feature correlates with left atrial pressure (LAP) as illustrated in FIG. 3. In particular, FIG. 3 is based on tracing of cardiogenic impedance and left atrial pressure during three rounds of rapid ventricular pacing. In FIG. 3, the left vertical axis represents left atrial pressure in mmHg, and corresponds to the solid line. The right vertical axis represents impedance, Ω, and corresponds to the dotted line. The horizontal axis represents time.

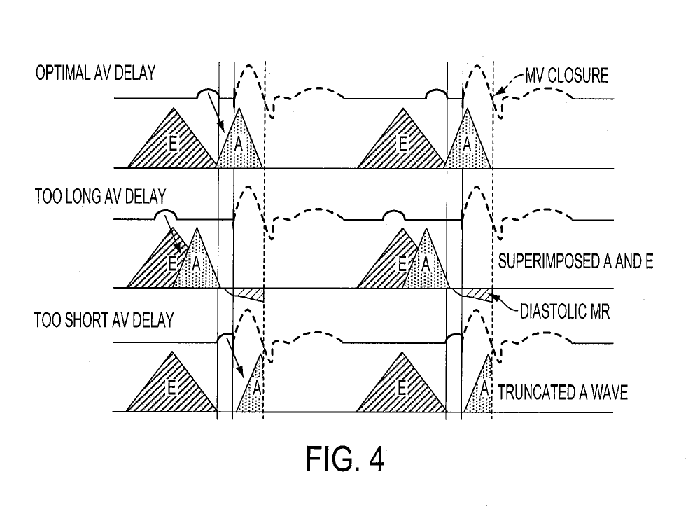

In some aspects of the disclosure, a method of calculating optimal paced/sensed AV, V-V, and LV-LV delays is described. The method uses intracardiac impedance signals to achieve device-based CRT optimization in CRT patients. In some aspects of the disclosure, cardiogenic impedance signals can be used to correlate with echo parameters such as E and A wave of Doppler echocardiographic recordings. The relationship of the E and A wave with the cardiac cycle is illustrated in FIG. 4. In particular, FIG. 4 is an exemplary schematic diagram showing the effect of atrioventricular (AV) delay on Doppler echocardiographic recordings of transmitral flow. The AV delay, for example, can be adjusted until the E wave, associated with a passive filling phase, and the A wave, associated with an active filling phase, are observed as separate peaks on a transmitral Doppler echocardiography image. In addition to evaluating the timing relationship between the E and A wave, the impedance amplitude and peak to peak intervals of other peaks can be evaluated for device timing optimization.

During left ventricular diastole, after the pressure drops in the left ventricle due to relaxation of the ventricular myocardium, the mitral valve opens, and blood travels from the left atrium to the left ventricle. About 70-80% of the blood that travels across the mitral valve occurs during the early filling phase of the left ventricle. This early filling phase is due to active relaxation of the ventricular myocardium, causing a pressure gradient that allows a rapid flow of blood from the left atrium, across the mitral valve. This early filling across the mitral valve is seen on a Doppler echocardiography of the mitral valve as the E wave. After the E wave, there is a period of slow filling of the ventricle.

Left atrial contraction or left atrial systole (during left ventricular diastole) causes added blood to flow across the mitral valve immediately before left ventricular systole. This late flow across the open mitral valve is seen on the Doppler echocardiography of the mitral valve as the A wave. The late filling of the LV contributes about 20% to the volume in the left ventricle prior to ventricular systole.

With an optimal atrioventricular interval, the mitral valve (MV) closes at the end of the A wave. If the atrioventricular delay is too long (as illustrated in the middle panel of FIG. 4), the E and A waves become fused and the diastolic filling is shortened. Late diastolic mitral regurgitation (MR) may then occur. If the atrioventricular delay is too short (as illustrated in the bottom panel of FIG. 4), the E and A waves become widely separated and the A wave is truncated by early mitral valve (MV) closure prior to completion of left ventricular filling.

In some aspects of the disclosure, a method calculates optimal or improved paced/sensed atrial ventricular (AV), interventricular (V-V), and/or intra-left ventricular (LV-LV) delay using cardiogenic impedance wave forms. The cardiogenic impedance data is based on sensing between various leads/electrodes. For example, the sensing can be from a right atrial (RA) lead to a left ventricular (LV) lead. If a quad pole lead (e.g., the lead 24 of FIG. 1) is used, any of the four sites can be selected. The cardiogenic impedance data can be based on sensing from the superior vena cava (SVC) electrode to the LV lead (again, any site on the lead). Other configurations derive the cardiogenic impedance data from sensing from a right ventricular (RV) lead to the LV lead (any site on the lead), and sensing between LV electrodes (if a multi site lead is used). Monitoring cardiogenic impedance waveforms associated with QRS onset (Bi-V pacing) can also facilitate the timing optimization. Additional vectors may also be analyzed. For example, SVC coil, RA (ring and tip) RV(coil, ring and tip), c an, the four LV electrodes, as well as any possible combination of endocardial, intracardial, and epicardial electrodes can be analyzed.

In general, excessively short AV delay (too short AV delay) can induce Cannon waves, and excessively long AV delay (too long AV delay) can induce diastolic mitral regurgitation. To avoid these limitations, it is desirable to establish an AV delay that optimizes blood flow in the heart. According to the present disclosure, the AV timing optimization using cardiogenic impedance can be achieved in accordance with a modified Ritter's method. For example, see Stanton Tony et al., “How should we optimize cardiac resynchronization therapy?” Eur Heart J. 2008 (Oct); 29(20):2458-2472, the disclosure of which is expressly incorporated by reference herein in its entirety. In other words, AV delay can be optimized using the equation as follows;

$$\begin{matrix}AVopt = AVshort + [ ( AVlong + QAlong ) - ( AVshort + QAshort ) ] \\ = AVlong - ( QAshort - QAlong )\end{matrix} \qquad ( 1 )$$

where:
AVshort and AVlong are the too short and too long AV delays, for example, 70-90 ms and 230-300 ms respectively;
QAshort is defined as the duration between the Bi-V pacing spike to the beginning of the LV systole (closure of the mitral valve) at excessively short programmed AV delay.
QAlong is defined as the duration between the end of the A wave (late mitral inflow) and the Bi-V pacing spike at excessively long programmed AV delay.

Rather than measuring QAshort and QAlong with echocardiography, the QAshort and QAlong parameters may be estimated based on cardiogenic impedance signals. The cardiogenic impedance data may be obtained using a CRT device such as the IMD device 10 in conjunction with various electrodes. In some aspects, the cardiogenic impedance signal is based on sensing from the RA lead to one of the electrodes on the LV lead. This vector is selected because it encompasses the mitral valve region, capturing the blood flow information across the mitral valve as well as the LV contraction information.

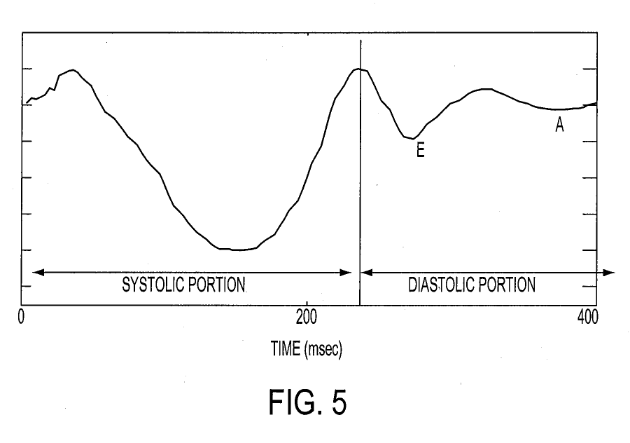

FIG. 5 shows an exemplary cardiogenic impedance signal using a RA-LV vector. The signal starts from the R wave and continues for the duration of a single cardiac cycle. According to aspects of the present disclosure, the echocardiographic E and A wave information is derived from the cardiogenic impedance signal.

When too long of an AV delay is programmed nonphysiologically, later Bi-V pacing induces fusion of the E and A waves (as seen in the middle section of FIG. 4). QAlong can thus be estimated by looking at the end of the A wave. More specifically, the time interval between maximum cardiogenic impedance in either RA to LV (any electrode) or SVC to LV (any electrode) vectors and Bi-V pacing spike can provide the QAlong.

When too short of an AV delay is programmed nonphysiologically, earlier Bi-V pacing truncates the A wave and abruptly changes the cardiogenic impedance morphology (as seen in the bottom section of FIG. 4). The time interval between the Bi-V pacing spikes and morphology changes in cardiogenic impedance in either RA to LV (any electrode) or SVC to LV (any electrode) vectors, especially quickly descending curvature, can provide the QAshort. Thus, the QAshort parameter can be estimated by taking the derivative of the systolic portion of the cardiogenic impedance signal and finding the maximum negative slope.

After calculating the optimal AV delay, the bi-ventricular (Bi-V) pacing percentage can be evaluated. If the Bi-V pacing percentage with the calculated optimal AV delay is below a threshold, for example 100% or at least 95%, for a period of time (e.g., 1-2 minutes), the calculated optimal AV delay may be adjusted (e.g., decreased 10 milliseconds), to improve the Bi-V pacing percentage to 100% or at least 95%, for example.

Another aspect of the present disclosure, selects which LV electrodes to use for accurate cardiogenic impedance calculation. Selecting an electrode may be useful when multiple electrodes are available on a lead, such as with the quad pole lead 24 of FIG. 1. The RA (or RV or SVC)-LV cardiogenic impedance waveforms can have varying features (e.g., morphology and amplitude) based on the LV electrode location. By selecting the appropriate LV electrode, cardiogenic impedance can be more accurately estimated, enabling improved setting of timing delay.

For RA (or RV or SVC)-LV cardiogenic impedance calculation, a LV electrode location that provides a waveform comparable to a typical echo-based E/A waveform is desirable. Some criteria for selecting the LV electrode are: 1) timing (i.e., activation during the diastolic period of a cardiac cycle) of E and A peaks; 2) morphology of E and A peaks (e.g., based on template matching); 3) consistency of cardiogenic impedance pattern (e.g., based on template matching or cycle to cycle matching); 4) signal to noise ratio (ensuring a reliable consistent morphology is present); and 5) random noise level (ensuring too much noise is not present). In order to select the LV electrode, the cardiogenic impedance waveform can be computed from the right atrial lead (or RV or SVC) to each LV electrode. The above mentioned criteria may be analyzed to select a suitable LV electrode for the use in timing optimization from a RA (or RV or SVC)-LV cardiogenic impedance calculation.

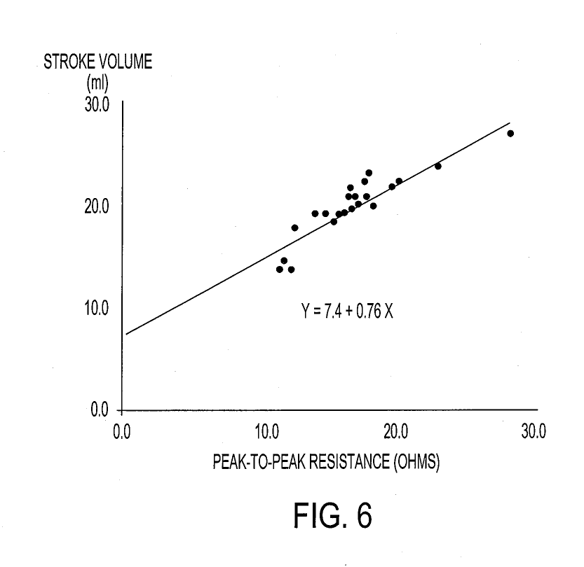

Once the electrode is selected and the AV delay is properly set, interventricular delay (V-V delay) optimization can be achieved by maximizing or improving stroke volume with cardiogenic impedance used as a surrogate. As is known, stroke volume measured by aortic flow linearly changes with peak to peak impedance as illustrated in FIG. 6. Therefore, peak-to-peak impedance across the heart's chambers can be an accurate and global surrogate for the measurement of stroke volume. That is, the amplitudes of the cardiogenic impedance wave form are analyzed. In an example of setting the V-V delay, the V-V delay is varied from 80 milliseconds to −80 milliseconds in some increment (e.g., 5 milliseconds). An optimal V-V delay is determined when peak to peak cardiogenic impedance (for example, based on either RVring to RVtip or RVtip to LVtip or RVtip to any LV electrode) is at a maximum level. In an example of setting the LV-LV delay, the programmable LV-LV delay is varied, for example up to 80 milliseconds, in some increment (e.g., 5 milliseconds). An improved or optimal LV-LV delay is determined using a similar method as programming V-V delay. In another embodiment for V-V delay and LV-LV delay optimization, the timing relationship of a maximum positive peak during the systolic portion (FIG. 5) and the E wave can be calculated. The goal for either V-V delay or LV-LV delay optimization is to prolong the time interval between the maximum positive peak during the systolic portion and the E wave without fusing or truncating the E and A waves. In another embodiment for V-V delay and LV-LV delay optimization, the time interval of the systolic portion will be monitored during the optimization algorithm. The goal is to prolong the systolic portion without fusing or truncating the E and A waves. When programming any one of three programmable delays (AV delay, V-V delay, and LV-LV delay), all of mentioned impedance features may be revisited to confirm other optimal delays remain the same, as they are related to each other.

The timing delays discussed above can be set at a time determined by the physician. For example, the timing delays can be set daily, weekly, monthly, or at some other interval, without visiting the physician.

The described methods may also be used to guide LV lead positioning at implant, for example by creating an impedance or stroke volume map during implant. The lead can be placed at a location that optimizes the stroke volume when stimulating.

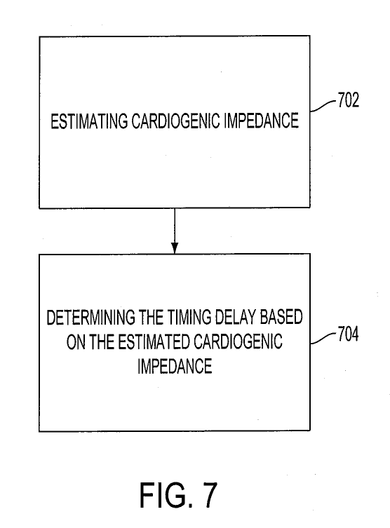

FIG. 7 illustrates an exemplary flowchart of a method for calculating a timing delay for an implantable medical device based on cardiogenic impedance. At block 702, cardiogenic impedance is estimated from a signal between a first electrode and a second electrode positioned in at least one chamber of a heart. At block 704 the timing delay is determined based on the estimated cardiogenic impedance.

An implantable medical device may have means for estimating and means for determining. In one aspect, the means may be the cardiogenic impedance module 123, the programmable microcontroller 60 and/or the memory 94. In another aspect, the aforementioned means may be a module or any apparatus configured to perform the functions recited by the aforementioned means.

Although the terms optimal and maximize have been used throughout this application, such language is not limiting. Rather, these terms are to be construed in a broader sense. For example, optimized or optimal also covers improved, better, enhanced, superior, etc. Similarly, maximize, also includes such terms increase, boost, enhance, etc.

The methodologies described herein may be implemented by various means depending upon the application. For example, these methodologies may be implemented in hardware, firmware, software, or any combination thereof. For a hardware implementation, the processing units, including programmable microcontroller 60 (FIG. 2) may be implemented within one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, electronic devices, other electronic units designed to perform the functions described herein, or a combination thereof.

For a firmware and/or software implementation, the methodologies may be implemented with modules (e.g., procedures, functions, and so on) that perform the functions described herein. Any machine or computer readable medium tangibly embodying instructions that may be in a form implantable or coupled to an implantable medical device may be used in implementing the methodologies described herein. For example, software code may be stored in a memory and executed by a processor. When executed by the processor, the executing software code generates the operational environment that implements the various methodologies and functionalities of the different aspects of the teachings presented herein. Memory may be implemented within the processor or external to the processor. As used herein the term “memory” refers to any type of long term, short term, volatile, nonvolatile, or other memory and is not to be limited to any particular type of memory or number of memories, or type of media upon which memory is stored.

The machine or computer readable medium that stores the software code defining the methodologies and functions described herein includes physical computer storage media. A storage medium may be any available medium that can be accessed by the processor of an implantable medical device. By way of example, and not limitation, such computer-readable media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. As used herein, disk and/or disc includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer readable media.

Although the present teachings and its advantages have been described in detail, it should be understood that various changes, substitutions and alterations can be made herein without departing from the spirit and scope of the present teachings as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present teachings, processes, machines, manufacture, compositions of matter, means, methods, or steps, presently existing or later to be developed that perform substantially the same function or achieve substantially the same result as the corresponding embodiments described herein may be utilized according to the present teachings. Accordingly, the appended claims are intended to include within their scope such processes, machines, manufacture, compositions of matter, means, methods, or steps.

What is claimed is:

1. A method of calculating a timing delay for an implantable medical device based on cardiogenic impedance, comprising:
estimating cardiogenic impedance from a signal between a first electrode and a second electrode positioned in at least one chamber of a heart; and
determining the timing delay based on the estimated cardiogenic impedance.

2. The method of claim 1, in which the timing delay comprises an atrio-ventricular (AV) delay.

3. The method of claim 2, in which estimating comprises calculating a maximum negative slope based on a derivative of a systolic portion of the signal.

4. The method of claim 2, in which estimating is based on a time interval between a bi-ventricular pacing spike and a maximum cardiogenic impedance.

5. The method of claim 2, further comprising evaluating a bi-ventricular pacing percentage associated with the timing delay to determine whether the bi-ventricular pacing percentage meets a threshold value.

6. The method of claim 1, in which the signal comprises a vector that encompasses a mitral valve region of the heart, the vector capturing information related to blood flow across the mitral valve region and information related to left ventricular contraction.

7. The method of claim 1, further comprising selecting the second electrode such that the signals acquired correlate to an echo-based E/A waveform.

8. The method of claim 7, in which the selection of the electrodes is based on at least one of timing, morphology of E and A peaks, consistency of a cardiogenic impedance pattern, signal to noise ratio and random noise level.

9. The method of claim 1, in which the timing delay comprises an interventricular timing delay.

10. The method of claim 9, in which the estimating comprises measuring peak to peak cardiogenic impedance.

11. An apparatus for calculating a timing delay for an implantable medical device based on cardiogenic impedance, comprising:
a memory; and
at least one processor coupled to the memory and configured:
to estimate cardiogenic impedance from a signal between a first electrode and a second electrode positioned in at least one chamber of a heart; and
to determine the timing delay based on the estimated cardiogenic impedance.

12. The apparatus of claim 11, in which the timing delay comprises an atrio-ventricular (AV) delay.

13. The apparatus of claim 12, in which the at least one processor is configured to estimate by calculating a maximum negative slope based on a derivative of a systolic portion of the signal.

14. The apparatus of claim 12, in which the at least one processor is configured to estimate based on a time interval between a bi-ventricular pacing spike and a maximum cardiogenic impedance.

15. The apparatus of claim 12, in which the at least one processor is further configured to evaluate a bi-ventricular pacing percentage associated with the timing delay to determine whether the bi-ventricular pacing percentage meets a threshold value.

16. The apparatus of claim 11, in which the signal comprises a vector that encompasses a mitral valve region of the heart, the vector capturing information related to blood flow across the mitral valve region and information related to left ventricular contraction.

17. The apparatus of claim 11, in which the at least one processor is further configured to select the second electrode such that the signals acquired correlate to an echo-based E/A waveform.

18. The apparatus of claim 17, in which the at least one processor is configured to select the electrodes based on at least one of timing, morphology of E and A peaks, consistency of a cardiogenic impedance pattern, signal to noise ratio and random noise level.

19. The apparatus of claim 11, in which the timing delay comprises an interventricular timing delay.

20. The apparatus of claim 19, in which the at least one processor is configured to estimate by measuring peak to peak cardiogenic impedance.

21. An apparatus for calculating a timing delay for an implantable medical device based on cardiogenic impedance, comprising:
means for estimating cardiogenic impedance from a signal between a first electrode and a second electrode positioned in at least one chamber of a heart; and
means for determining the timing delay based on the estimated cardiogenic impedance.

22. A computer program product for calculating a timing delay for an implantable medical device based on cardiogenic impedance, comprising:
a computer-readable medium having non-transitory program code recorded thereon, the program code comprising:
program code to estimate cardiogenic impedance from a signal between a first electrode and a second electrode positioned in at least one chamber of a heart; and
program code to determine the timing delay based on the estimated cardiogenic impedance.
